(19) United States
(12) Patent Application Publication
(10) Pub. No.: US 2016/0299093 A1
(43) Pub. Date: Oct. 13, 2016

(54) DIAGNOSTIC METHOD AND DEVICE FOR ASSESSING HUMAN JOINT FLUID REACTIVITY TO CoCrMo ALLOY

(71) Applicant: Jeremy Gilbert, Fayetteville, NY (US)

(72) Inventor: Jeremy Gilbert, Fayetteville, NY (US)

(73) Assignee: Syracuse University, Syracuse, NY (US)

(21) Appl. No.: 15/100,651

(22) PCT Filed: Dec. 2, 2014
(86) PCT No.: PCT/US2014/068101

§ 371 Date: Jun. 1, 2016

Related U.S. Application Data
(60) Provisional application No. 61/910,643, filed on Dec. 2, 2013.

(51) Int. Cl. G01N 27/02; G01N 27/04; G01N 27/22; A61B 5/00

(52) U.S. Cl. CPC G01N 27/026; A61B 5/411; G01N 27/041; G01N 27/22

(57) ABSTRACT

A chamber for testing the reactivity of patient fluids to a metallic implant alloy, such as CoCrMo, and a method of determining the reactivity of the patent to the alloy. A sealed mini-electrochemical cell incorporates the alloy surface as well as reference and counter electrodes coupled to a potentiostat for performing electrochemical measurements of the alloy surface while it is in direct contact with freshly harvested human fluids. Freshly obtained body fluids are thus assessed in terms of their bioelectrochemical reactivity with the alloy surface as a measure of the likely reactivity of the patient to the alloy if it were to be implanted into the patient.

### BACKGROUND OF THE INVENTION

1. Field of the Invention

The present invention relates to medical implants and, more specifically, to a testing apparatus for measuring patent reactivity to metallic biomaterials such as CoCrMo alloys used in an implant.

2. Description of the Related Art

Metallic alloys, such as Cobalt-Chromium-Molybdenum (CoCrMo) alloys, are used widely in medical devices implanted into the human body. In some circumstances these alloys can stimulate a significant inflammatory and/or immune response, particularly when ions or particles are released. Phagocytic cells of the body can directly release reactive oxygen species, like hydrogen peroxide, into the local fluid spaces around the implant in reaction to the presence of the alloy or its degradation products. Recent electrochemical experiments have shown that CoCrMo corrosion behavior is highly sensitive to the presence and concentration of even small amounts of reactive oxygen species. Additions of as little as 100 uM H₂O₂ to physiological solutions significantly alters the corrosion potential of the alloy in a rapid and systematic way, and also significantly reduces the impedance of the surface and raises the corrosion rate of the CoCrMo in proportion to the amount of reactive species present. In many total joint replacements that are suffering severe inflammatory reactions by the body, there is a local build-up of solution based inflammatory species that reflect the severity of the biological reaction.

Presently, the only assessments available for patient reactivity to metals is by using allergy challenge tests on the skin. These are highly variable methods that often are not able to detect sensitivity to metals. As a result, no direct method is available and there is a need for a mechanism to directly measure the level of reactive species present in a patient to act as a diagnostic tool for physicians in assessing patient-implant sensitivity and reactivity.

### BRIEF SUMMARY OF THE INVENTION

The present invention comprises a testing apparatus that can be used to assess the reactivity of human biological fluids, such as those obtained from reactive joint spaces, on metallic biomaterials. For example, testing apparatus may be used to determine if a patient's body is reacting aggressively to the implantation of a metal alloy, such as CoCrMo, used in hip and knee replacements. Testing apparatus comprises a sealed mini-electrochemical corrosion cell that incorporates a metallic biomaterial surface, a reference electrode, and a counter electrode, that allow electrochemical measurement of the alloy surface after it is placed in direct contact with freshly harvested human fluids. The alloy surface, as well as the electrodes, are connected to a potentiostat / Electrochemical Impedance Spectroscopy (EIS) system for taking appropriate measurements and readings and outputting results so that freshly obtained body fluids from the joint space, for example, can be assessed in terms of their bioelectrochemical reactivity with the alloy as a measure of the patient's likely reactivity to the alloy. The more intense the immune or inflammatory response of the patient, the larger will be the changes in electrochemical activity of the alloy surface that can be measured and converted into a scale of reactivity.

The testing apparatus provides a means to diagnose and assess patient reactivity in the patient's local tissues to the alloy surface either before or after implantation with the alloy based implants. The electrochemical reactivity can be measured in one of three ways: (i) by the voltage developed, (ii) by the impedance of the metal, or (iii) by the rate at which the metal corrodes. In each case, the more reactive the body fluid is, the greater the reactivity of the CoCrMo alloy surface, for example, will be when testing with the fluid in the testing apparatus. In use, a doctor may obtain a sample of human fluid from the region where the implant is located (or will be located). The body fluid is then transferred into mini-corrosion cell by inserting a needle through a sealing cap on the cell. When the alloy surface comes into contact with fluid, reactions between the alloy surface and the fluid are measured and then used to determine the sensitivity of the patient to the metal.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a testing apparatus according to the present invention;

FIG. 2 is a graph showing the effect of hydrogen peroxide on Open Circuit Potential (OCP) measurements of CoCrMo;

FIG. 3 is a graph of OCP versus H₂O₂ concentration;

FIG. 4 is a graph of the effect of H₂O₂ on the impedance of CoCrMo;

FIG. 5 is a graph of the polarization behavior of CoCrMo as a function of hydrogen peroxide and pH levels;

FIG. 6 is a graph of OCP measurements over time for different solutions;

FIG. 7 is a graph of Polarization tests results of CoCrMo in different solutions;

FIG. 8 is a Bode plot for CoCrMo in all solutions tested; and

FIG. 9 is a graph of the effect of Fenton's reagent on OCP; and

FIG. 10 is a graph of voltage in the transpassive region in response to the introduction of Fenton's reagent.

### DETAILED DESCRIPTION OF THE INVENTION

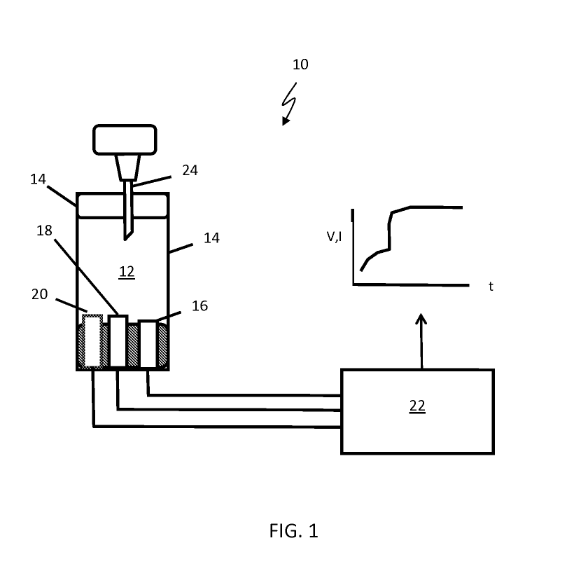

Referring now to the drawings, wherein like reference numerals refer to like parts throughout, there is seen in FIG. 1 a testing apparatus 10 for assessing the reactivity of human biological fluids 12, such as those obtained from reactive joint spaces. Testing apparatus 10 is used to determine if a patient's body is reacting aggressively to the implantation of a metal alloy, such as CoCrMo, used in hip and knee replacements. Testing apparatus 10 comprises a sealed mini-electrochemical corrosion cell 14 that incorporates a CoCrMo alloy surface 16, a reference electrode 18, and a counter electrode 20, that allow electrochemical measurement of CoCrMo alloy surface 16 after it is placed in direct contact with freshly harvested human fluids 12. Surface 16, as well as electrodes 18 and 20, are connected to a potentiostat/Electrochemical Impedance Spectroscopy (EIS) system 22 for taking appropriate measurements and readings and outputting results. Mini-corrosion cell 14 allows freshly obtained body fluids from the joint space, for example, to be assessed in terms of their bioelectrochemical reactivity with CoCrMo as a measure of the patient's reactivity to the alloy. The more intense the immune or inflammatory response of the patient, the larger will be the changes in electrochemical activity of CoCrMo alloy surface 16 that can be measured and converted into a scale of reactivity.

Mini-corrosion cell 14 provides a means to diagnose and assess patient reactivity in the patient's local tissues to CoCrMo alloy surface 16 either before or after implantation with CoCrMo alloy based implants. The electrochemical reactivity can be measured in one of three ways: (i) by the voltage developed, (ii) by the impedance of the metal, or (iii) by the rate at which the metal corrodes. In each case, the more reactive that body fluid 12 is likely to be in situ, the greater the electrochemical reactivity of CoCrMo alloy surface 16 will be with fluid 12 in cell 14.

In use, a doctor may obtain a sample of human fluid 12 from the region where the implant is located (or will be located). Body fluid 12 is then transferred into mini-corrosion cell 14, such as by a needle 24 inserted through a sealing cap 26 on cell 14. When CoCrMo alloy surface 16 comes into contact with fluid 12, reactions between the CoCrMo alloy surface 16 and fluid 12 can be used to determine the sensitivity of the patient to the metal. As seen in FIG. 1, mini-corrosion cell 14 can be configured to directly receive body fluids by injection via needle 24 and be sterilizable so that cell 14 will keep fluid 12 sterile and isolated from the outside in the event fluid 12 is infected. Mini-corrosion cell 14 could be disposable, treatment (e.g., injection of bleach or other chemical to kill the biological species), could be disassembled, cleaned and reused.

CoCrMo alloy surfaces were prepared for electrochemical analysis by polishing the surfaces to 600 grit and then placing them into an electrochemical cell containing phosphate buffered saline (PBS, a simplified analog of human fluids). A potentiostat (Solartron 1280C, Solartron Analytical) was used for electrochemical measurements. All electrochemical tests were made through a three electrode system with a reference electrode (Ag/AgCl), a counter electrode (Carbon rod) and a working electrode (CoCrMo disk). Open Circuit Potential (OCP) were monitored for 40 minutes for 4 different solutions, namely, phosphate buffered saline (PBS) solution (pH 7.4); PBS with 30 mM H₂O₂; PBS with HCl (pH 3) solutions and 30 mM H₂O₂ ; and PBS with HCl (pH 3). These solutions were used for polarization test and Electrochemical Impedance Spectroscopy (EIS) tests. Samples were brought in contact with the electrolyte solution for 15 minutes before polarization test with a starting and final potential of −1V (vs. Ag/AgCl), vertex potential of +1V (vs. Ag/AgCl) and a scan rate of 1 mV/s. EIS measurements were carried out at OCP immediately after solution was added. A 10 mv voltage was applied to the interface while varying the frequency of input voltage from 20 KHz to 10 mHz.

In a first test, the open circuit potential of the CoCrMo alloy is monitored by comparing the voltage of the alloy to a reference electrode voltage that remains constant. The PBS solution bathing the CoCrMo is modified by the addition of small amounts of hydrogen peroxide and the Open Circuit Potential (OCP) is monitored over time. The range of concentrations was from 100 uM to 30 mM hydrogen peroxide. That is, hydrogen peroxide was added to the PBS at a specific time such that the concentration of the hydrogen peroxide was known. The OCP was then monitored over 1 h of time. In a second test, both hydrogen peroxide and hydrochloric acid were added to the PBS solution and the OCP of the CoCrMo was monitored with pH ranges from 7.4 to 1 were assessed. In a final test, the impedance characteristics of the CoCrMo was measured using electrochemical impedance spectroscopy methods under varying conditions of hydrogen peroxide and pH changes to the solution. In these experiments, a small oscillating voltage was applied to the CoCrMo and the current response (amplitude and phase) are measured to determine the resistive and capacitive behavior of the surface. These can then be used to assess how the solution reactivity affects the CoCrMo behavior in a systematic way.

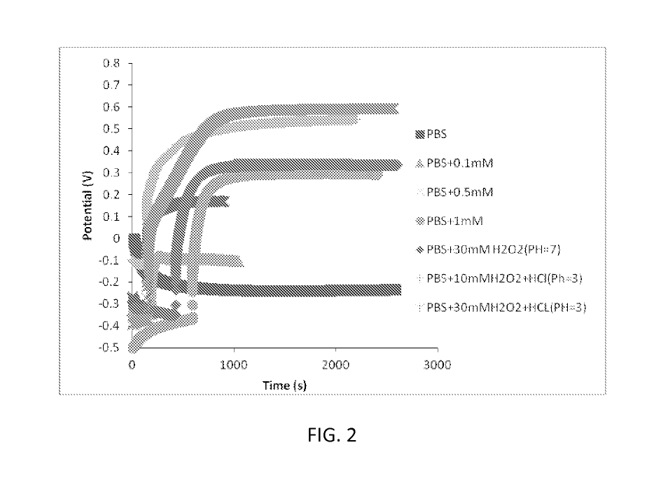

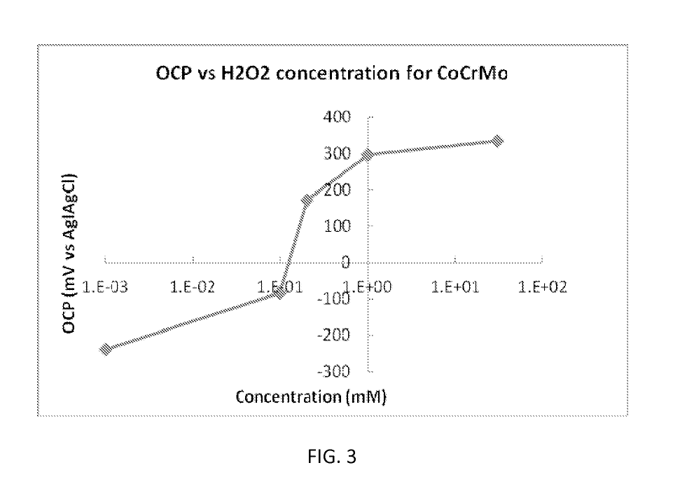

The effect of hydrogen peroxide on OCP measurements of CoCrMo are seen in FIG. 2. Addition of hydrogen peroxide results in a rapid and immediate change in the OCP of the alloy. In all cases the OCP rises to a more positive potential in direct proportion to the amount of reactive oxygen species present. Even at 100 uM H2O2, the OCP increased about 200 mV in less than 200 s. This rapid and significant change in potential may be used to assess the severity of the interaction between solution and alloy. Additional effects are seen when the pH of the solution is made more acidic (lower). Voltage increases up to 600 mV can be seen in FIG. 2. Plotting the final OCP versus H₂O₂ concentration is shown in FIG. 3. Note, the lowest concentration is for PBS, but is plotted at 10-3 mM concentration on the log scale.

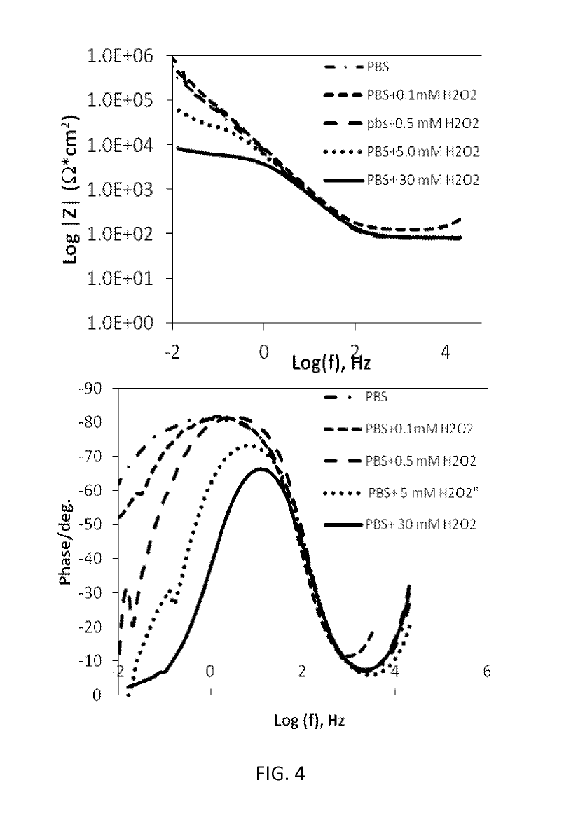

The effect of H₂O₂ on the impedance of CoCrMo is seen in FIG. 4. These data show clear decreases in low frequency impedance as the hydrogen peroxide concentration increases. Similarly, the phase angle behavior systematically changes as well. Both signals are highly sensitive to the concentration and may be capable of being utilized in a quantitative fashion for analytical purposes.

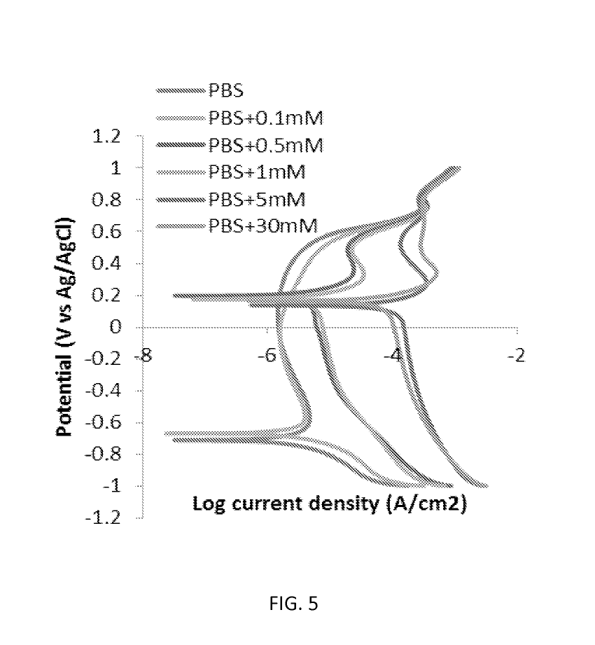

The polarization behavior of CoCrMo as a function of hydrogen peroxide and pH levels is seen in FIG. 5. These results show that small additions of hydrogen peroxide significant alter the polarization behavior. Changes in both the anodic and cathodic reactions present alter where the equilibrium potential occurs and raises the corrosion currents seen with increasing peroxide content.

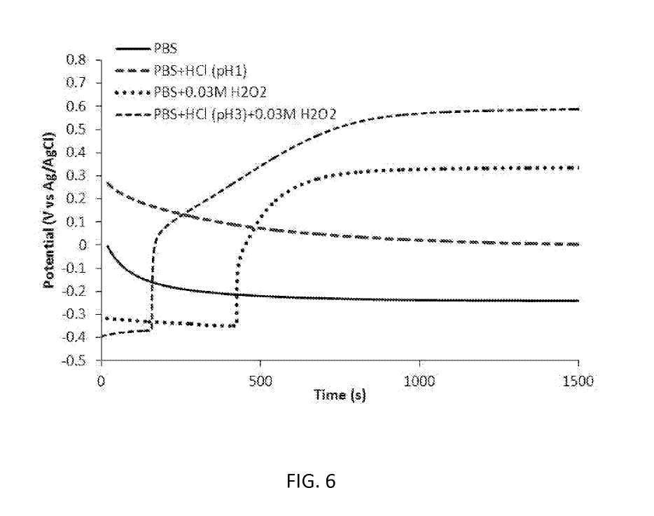

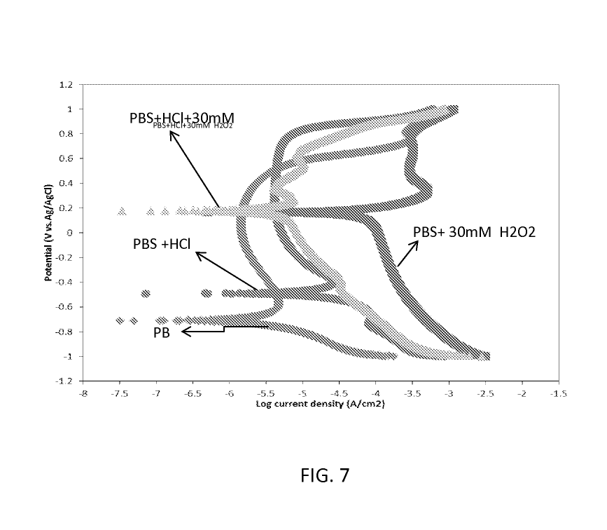

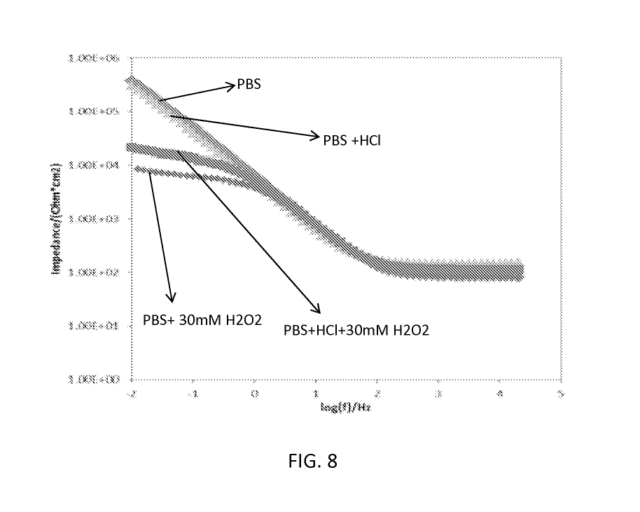

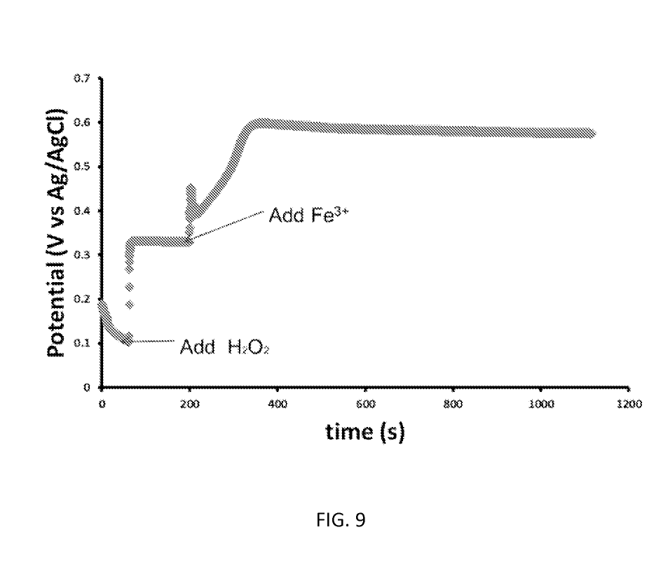

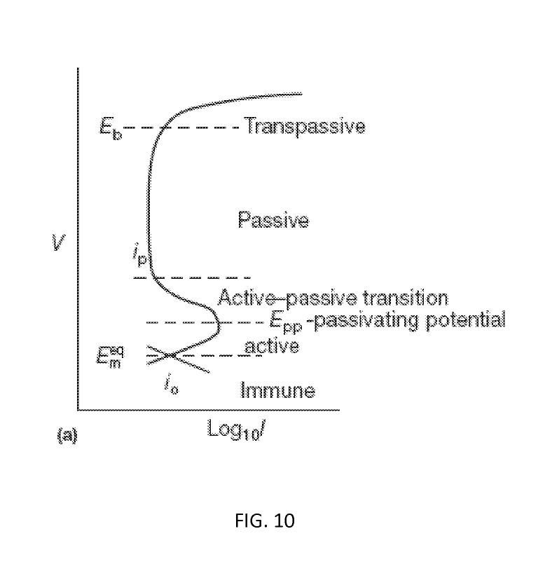

These results show that CoCrMo alloy is highly sensitive to the presence of reactive oxygen species, as would be present in inflamed solution surrounding CoCrMo hip implants. Small changes in solution composition yield large and systematic changes in CoCrMo that can be used as a diagnostic tool for assessing inflammatory index of patients with CoCrMo implants. More specifically, OCP, polarization tests and EIS results show that the corrosion behavior of CoCrMo alloy is largely affected by small amount presence of H₂O₂. OCP shifts positively from −0.35 V to 0.35V and 0.6V (vs Ag/AgCl) with 30 mM addition of H₂O₂ into PBS or PBS with HCl (pH 3) solutions respectively, as seen in FIG. 6. In the 0.6V (vs Ag/AgCl) range, the chromium oxide can become transpassive and the Cr⁶⁺ may be released. The polarization plots show that with 30 mM addition of H₂O₂ into PBS solution, the corrosion current density rises more than 40 times as seen in FIG. 7. With HCl (pH 3) present, the corrosion current increases 10 and 5 times from the PBS or the PBS with 30 mM H₂O₂ respectively. The impedance results indicate that the oxide impedance decreases with the addition of 30 mM H₂O₂ into PBS or PBS with HCl (pH 3), as seen in FIG. 8. At low frequency (0.01 Hz), the presence of 30 mM H₂O₂ decreases the impedance value around 100 times from the PBS only case, and 30 times for the PBS with HCl solution. Thus, corrosion susceptibility of CoCrMo alloy is significantly altered when PBS or PBS with HCl are modified with hydrogen peroxide. Increase of OCP to 0.6V range might oxidize the Cr ion into the +6 state. With H₂O₂ present, corrosion current density rises and oxide impedance decreases in PBS or PBS with HCl (pH=3). Furthermore, as seen in FIG. 9 and FIG. 10, the introduction of PBS and Fenton's reagent at pH 7.4 (0.1 mM Fe3+ and 10 mM H₂O₂), which replicates another aspect of the reactive chemical species environment involved in inflammatory cell induced corrosion, also results in a significant change in OCP over time with voltage levels entering the transpassive region.

Based on these tests, a sample of patient fluid that results in one or more of the electrochemical responses when introduced into cell 14 is indicative of a patient having a likelihood of an adverse reaction. Preferably, a scale of inflammatory index can be developed where increases in OCP reflect increases in the oxidizing potential of the solution and thus demonstrate a more reactive impact of the patient's fluid on the alloy. Alternatively, the impedance of the alloy surface as a function of the fluid chemistry may be used as lower impedances reflect a high inflammatory or oxidizing capability of the joint fluid being tested. Typical impedances measured for CoCrMo and Ti alloys are in the 30,000 to 10,000,000 ohm-cm2 range in PBS. Thus, drops in impedance, such as a decrease in the impedance of the surface up to 100 times the value in PBS, can be used to determine the alloy's sensitivity to the joint fluid attack.

While the present invention was demonstrated using a CoCrMo alloy surface, other metallic alloys, such as 316L stainless steel and various titanium alloys, may be evaulated using the apparatus and method of the present invention. For example, titanium alloys such as NiTi shape memory alloys, CP-Ti, Ti-6Al-4V, Ti-6Al-7Nb, can be used as alloy surface 16 and be exposed to bodily fluid to evaluate the electrochemical response. While the electrochemical response that corresponds to a risk of an adverse reaction may differ for each alloy, the response that corresponds to the of an adverse reaction can be determined by performing the same tests as those depicted in FIGS. 2-10 and discussed above to determine the specific manner in which a particular alloy will respond to the presence of highly reactive species in a sample of joint fluid.

What is claimed:

1. A testing apparatus for determining whether a patient may have sensitivity to a metallic medical implant, comprising:
a electrochemical cell defining a chamber therein and having a port through which a sample of fluid may be introduced into the chamber;
a metallic biomaterial alloy surface positioned in the chamber;
a reference electrode positioned in the chamber;
a counter electrode positioned in the chamber; and
a potentiostat coupled to the metallic biomaterial alloy surface, the reference electrode, and the counter electrode.

2. The apparatus of claim 1, wherein the metallic biomaterial alloy surface is a cobalt-chromium molybdenum (CoCrMo) alloy.

3. The apparatus of claim 1, wherein the metallic biomaterial alloy surface is a titanium alloy.

4. The apparatus of claim 3, wherein the titanium alloy is selected from the group consisting of NiTi shape memory alloy, CP-Ti, Ti-6Al-4V and Ti-6Al-7Nb.

5. The apparatus of claim 1, wherein the potentiostat is configured to measure the open circuit potential of the CoCrMo alloy surface.

6. The apparatus of claim 1, wherein the potentiostat is configured to measure the impedance characteristics of the CoCrMo alloy surface.

7. The apparatus of claim 1, wherein the potentiostat is configured to measure the polarization of the CoCrMo alloy surface.

8. A method of determining whether a patient may have sensitivity to a medical implant, comprising the steps of:
collecting a sample of fluid from a patient;
providing an electrochemical cell defining a chamber therein and having a port through which the sample of fluid may be introduced into the chamber, a CoCrMo alloy surface positioned in the chamber, a reference electrode positioned in the chamber, a counter electrode positioned in the chamber, and a potentiostat coupled to the CoCrMo alloy surface, the reference electrode, and the counter electrode;
inserting the sample of fluid through the port of the electrochemical cell into contact with the CoCrMo alloy surface;
measuring the electrochemical response of the CoCrMo alloy surface to the fluid over time; and
determining whether the patient may have an adverse reaction to a CoCrMo alloy implant based upon the electrochemical response of the CoCrMo alloy surface to the fluid of the patient.

9. The method of claim 8, wherein the step of measuring the electrochemical response of the CoCrMo alloy surface to the fluid over time comprises measuring the open circuit potential of the CoCrMo alloy surface.

10. The method of claim 9, wherein the step of determining whether the patient may have an adverse reaction to a CoCrMo alloy implant is based on whether the open circuit potential becomes more positive when the sample of fluid is placed into contact with the CoCrMo alloy surface.

11. The method of claim 8, wherein the step of measuring the electrochemical response of the CoCrMo alloy surface to the fluid over time comprises measuring the impedance of the CoCrMo alloy surface.

12. The method of claim 8, wherein the step of determining whether the patient may have an adverse reaction to a CoCrMo alloy implant is based on whether the impedance decreases logarithmically when the sample of fluid is placed into contact with the CoCrMo alloy surface.

13. The method of claim 8, wherein the step of measuring the electrochemical response of the CoCrMo alloy surface to the fluid over time comprises measuring the polarization of the CoCrMo alloy surface.

14. The method of claim 8, wherein the step of determining whether the patient may have an adverse reaction to a CoCrMo alloy implant is based on whether the polarization increases when the sample of fluid is placed into contact with the CoCrMo alloy surface.
